(19) United States
(12) Patent Application Publication
(10) Pub. No.: US 2025/0032110 A1
(43) Pub. Date: Jan. 30, 2025

(54) RETRACTORS

(71) Applicant: SURE RETRACTORS LIMITED, Glasgow (GB)

(72) Inventor: Forbes BUTTERFIELD, Glasgow (GB)

(21) Appl. No.: 18/715,092

(22) PCT Filed: Dec. 13, 2022
(86) PCT No.: PCT/GB2022/053209

§ 371 Date: May 30, 2024

(30) Foreign Application Priority Data
Dec. 14, 2021 (GB) 2118086.4

(51) Int. Cl. A61B 17/02; A61B 17/00; A61L 31/08; A61L 31/10; A61L 31/16

(52) U.S. Cl. CPC A61B 17/0206; A61L 31/088; A61L 31/10; A61L 31/16; A61B 2017/00526; A61B 2017/00893; A61L 2400/04; A61L 2420/02

(57) ABSTRACT

The surgical retractor (10) comprises a support (12), a first arm (14) extending from the support, and a second arm (16) extending from the support, the first and second arms extending from a same side of the support and spaced apart from each other along the support. The surgical retractor (10) further comprises a first anatomy engaging member (42) having a first interior face (52) and a first exterior face (50), and a second anatomy engaging member (44) having a second interior face (52) and a second exterior face (50). The first anatomy engaging member (42) depends along its length from the first arm (14) whereby the first exterior face (50) faces away from the second anatomy engaging member (44). The second anatomy engaging member (44) depends along its length from the second arm (16) whereby the second exterior face (50) faces away from the first anatomy engaging member (42). Each of the first and second anatomy engaging members (42), (44) is formed from a plastics material. Each of the first exterior face (50) and the second exterior face (50) has a coating thereon, the coating comprising at least one haemostatic agent.

### FIELD OF THE INVENTION

The present invention relates to surgical retractors, methods of making such surgical retractors, and methods of surgery involving such surgical retractors.

### BACKGROUND ART

Surgical retractors are known. Surgical retractors separate the edges of an incision or wound, and perhaps also hold back underlying organs and tissues, to allow better visibility of and access to anatomy under the incision or wound.

The present inventors have become appreciative of shortcomings of known surgical retractors. The present invention has been devised in light of the inventors' appreciation of such shortcomings. It is therefore an object for the present invention to provide an improved surgical retractor comprising a support, first and second arms extending from the support, and first and second anatomy engaging members depending from the first and second arms respectively.

It is a further object for the present invention to provide a method of making a surgical retractor comprising a support, first and second arms extending from the support, and first and second anatomy engaging members depending from the first and second arms, respectively. It is a further object for the present invention to provide a method of surgery on a human or animal subject involving use of a surgical retractor which comprises a support, first and second arms extending from the support, and first and second anatomy engaging members depending from the first and second arms, respectively.

### Statement of Invention

According to a first aspect of the present invention there is provided a surgical retractor comprising:
a support;
a first arm extending from the support;
a second arm extending from the support, the first and second arms extending from a same side of the support and spaced apart from each other along the support;
a first anatomy engaging member having a first interior face and a first exterior face; and
a second anatomy engaging member having a second interior face and a second exterior face, wherein
the first anatomy engaging member depends along its length from the first arm whereby the first exterior face faces away from the second anatomy engaging member;
the second anatomy engaging member depends along its length from the second arm whereby the second exterior face faces away from the first anatomy engaging member;
each of the first and second anatomy engaging members is formed from a plastics material; and
each of the first exterior face and the second exterior face has a coating thereon, the coating comprising a haemostatic agent.

The surgical retractor comprises a support and first and second arms. The first and second arms extend from a same side of the support and are spaced apart from each other along the support. The surgical retractor comprises first and second anatomy engaging members. The first anatomy engaging member has a first interior face and a first exterior face, and the second anatomy engaging member has a second interior face and a second exterior face. The first anatomy engaging member depends along its length from the first arm whereby the first exterior face faces away from the second anatomy engaging member. The first anatomy engaging member may therefore depend from the first arm in a longitudinal direction of the first anatomy engaging member. The second anatomy engaging member depends along its length from the second arm whereby the second exterior face faces away from the first anatomy engaging member. The second anatomy engaging member may therefore depend from the second arm in a longitudinal direction of the second anatomy engaging member.

Each of the first and second anatomy engaging members is formed from a plastics material. Furthermore, each of the first exterior face and the second exterior face has thereon the coating comprising a haemostatic agent. Plural different haemostatic agents may be comprised in the coating.

Haemostasis refers to the normal physiological process, i.e. intrinsic process, by which loss of blood from damaged blood vessels is reduced. Haemostasis has three main steps: vasoconstriction; temporary blockage by a platelet plug of a break in a blood vessel; and blood coagulation. A surgical retractor is inserted into an incision or wound which may have been made or sustained recently whereby the incision or wound is still bleeding. A haemostatic agent can promote and/or support the normal physiological process of haemostasis at one or more of the three main steps of haemostasis. Early haemostasis is desirable for wound healing and patient health. The present inventors have realised that surgical retractors have a positive role to play in haemostasis. Blood oozing from tissue can obscure the surgeon's view of the incision or wound site. Alternatively or in addition, blood oozing from tissue can make surgery more difficult. Aside from these problems, blood loss may be detrimental to the patient. Promoting and/or supporting haemostasis by way of the surgical retractor may therefore be desirable. The exterior face of each of the first and second anatomy engaging members therefore has thereon a coating comprising a haemostatic agent.

In view of the first and second exterior faces pressing against tissue and thereby being liable to hinder healing or perhaps even cause injury to the tissue, the first and second exterior faces have thereon a coating comprising a haemostatic agent. In addition, the first and second interior faces may have thereon a coating comprising a haemostatic agent. Having a haemostatic agent comprising coating on the interior and exterior faces of the first and second anatomy engaging members may be beneficial in certain circumstances, such as when bleeding is not only from the tissues or organs being retracted but is more general.

The haemostatic agent may be self-supporting. Some haemostatic agents, such as chitosan which is described below, may be capable of being formed as fibres whereby such a haemostatic agent may be applied directly to the anatomy engaging member to form a coating thereon.

As described above, the haemostatic agent is comprised in a coating on the face of the anatomy engaging member.

Depending on the nature of the haemostatic agent and how the coating is to be applied to the anatomy engaging member, the haemostatic agent may be dissolved in an appropriate solvent or may be dispersed in an appropriate medium of suspension to form a colloid. A haemostatic mixture comprising dissolved or dispersed haemostatic agent may thus be formed ready for application to an anatomy engaging member.

The haemostatic mixture may be applied to the anatomy engaging member to form the haemostatic coating by spraying the anatomy engaging member with the haemostatic mixture. Alternatively the anatomy engaging member may be immersed in the haemostatic mixture. Spray coating may be preferred over immersion (or dip coating) when the haemostatic mixture is applied to an anatomy engaging member formed of plastics material. Furthermore, spray coating may be effective for application of chitosan. Otherwise, spray coating has been found to be an easy, versatile and efficient process. The coating on the anatomy engaging member may then be dried, for example by heating at between 60 to 80 degrees Centigrade.

The haemostatic agent may be applied to the anatomy engaging member by hot or cold plasma spraying.

The haemostatic agent may be applied to the anatomy engaging member by chemical vapor deposition.

Adhesion of the coating to the anatomy engaging member may be improved by increasing surface roughness of the anatomy engaging member. The anatomy engaging member may therefore be treated to increase surface roughness before application of the coating. Surface roughness treatment may comprise plasma treatment which is preferred over other roughness treatments because it is non-toxic and residues free. The plasma treatment may comprise application of at least one of oxygen, argon, and air plasma.

The coating on the anatomy engaging member may comprise plural different haemostatic agents. The plural different haemostatic agents may stimulate haemostasis in respectively different ways. For example, the coating on the anatomy engaging member may comprise chitosan as a first haemostatic agent, and kaolin as a second haemostatic agent.

The haemostatic agent may be operative to stimulate the intrinsic coagulation pathway. The haemostatic agent may therefore comprise at least one of a starch and a clay mineral. The clay mineral may belong to the kaolin group or the smectite group. Where the clay mineral belongs to the kaolin group, the haemostatic agent may be a silicate mineral and more specifically an aluminosilicate mineral. The aluminosilicate mineral may be comprised in kaolin.

The haemostatic agent may be operative to promote formation of a temporary blockage in a blood vessel. This may be achieved by promoting cross-linking of red blood cells. The haemostatic agent may therefore comprise at least one organic compound and more specifically a polysaccharide. The polysaccharide may be cellulose or chitosan. Chitosan may be a preferred haemostatic agent because homogeneous chitosan comprising coatings may be readily formed by spray or dip coating and in particular spray coating. Furthermore chitosan is compatible with sterilisation by ethylene oxide.

The haemostatic agent may be operative to promote vasoconstriction. The haemostatic agent may therefore comprise at least one astringent, such as anhydrous aluminium sulfate.

The haemostatic agent may comprise a coagulation factor such as thrombin.

The first and second exterior faces may be generally and more specifically substantially oppositely directed. In view of the first and second arms being spaced apart from each other along the support, the first and second interior faces may be spaced apart from each other.

The anatomy engaging member may comprise a main body which defines the respective exterior and interior faces. The main body may have the form of an elongate plate. The main body may be generally and more specifically substantially rectangular when viewed from the interior or exterior face.

As described above, the first and second anatomy engaging members depend from the first and second arms, respectively. Furthermore, at least one of the first and second anatomy engaging members may be attached to the respective arm. More specifically the anatomy engaging member may be attached to the respective arm for movement, such as sliding movement, of the anatomy engaging member along the arm.

The anatomy engaging member may be configured such that it extends substantially orthogonally to the respective arm. The anatomy engaging member may be configured to attach releasably to a respective arm.

The anatomy engaging member may be integrally formed. As described above, the anatomy engaging member is formed from a plastics material. The plastics material may comprise polyamide and more specifically a partially aromatic polyamide, such as polyarylamide, such as GS Ixef® 1022 from Solvay S. A. The anatomy engaging member may therefore be suited to single use.

Alternatively or in addition, the plastics material may comprise polyester, and more specifically a polycarbonate. The inventors have found polycarbonate to provide a coefficient of friction which is advantageous in certain applications. Aside from coefficient of friction, stiffness of the plastics material has been discovered to be of significance under certain circumstances. Surgical procedures involving retraction may be of considerable duration. Retracted tissue may therefore be subject to distress over a long period. Tissue damage and perhaps even necrosis may therefore be a risk. The inventors have found some degree of flexure of the anatomy engaging member to be desirable in reducing likelihood of tissue distress. A polyester material, and more specifically a polycarbonate material, has been found to have appropriate flexibility.

Distress caused by the anatomy engaging member pressing unduly against tissue may be indicated by blanching of the tissue. However, known anatomy engaging members obscure tissue blanching from clinician observation. The anatomy engaging member may therefore be formed at least in part from material, and more specifically plastics material, that is transparent in the sense of permitting transmission of light therethrough. The clinician may therefore be able to observe blanching of tissue underneath the anatomy engaging member. Aside from providing for ease of observation of tissue blanching, transparent material may provide for ease of determination of cleanliness of the anatomy engaging member, such as by observation by a clinician. The anatomy engaging member may be formed from a transparent polycarbonate (PC) material, such as Makrolon® 2458.

Each of the first and second arms may extend from a respective location, the two locations spaced apart from each other along the support. The support may be elongate and the first and second arms may extend from respective locations on the elongate support, the respective locations spaced apart from each other along the longitudinal direction of the elongate support.

The first and second arms may be mechanically coupled to the support whereby separation between the first and second arms is substantially fixed. More specifically, the first and second arms may be attached to the support so that the first and second arms do not move along the support. Such attachment may not exclude movement of at least one of the first and second arms relative to the support but such that separation between the first and second arms is substantially fixed. For example, at least one of the first and second arms may be rotatable around the support.

Alternatively, the surgical retractor may be configured for change in separation between the first and second arms along the support, such as by way of clinician operation, whereby separation between the first and second exterior faces is changed. Change in separation between the first and second arms may be by movement of at least one of the first and second arms along the support whereby there is change in extent of spacing apart of the first and second arms along the support. In use, separation between the first and second arms of the surgical retractor may be set to allow the first and anatomy engaging members to be received readily in an incision or wound. The clinician may then increase separation between the first and second arms whereby the first exterior face of the first anatomy engaging member bears against a first side of the incision or wound and the second exterior face of the second anatomy engaging member bears against a second, opposite side of the incision or wound. The incision or wound may thus be retracted.

The support may be elongate. The first and second arms may be attached at first and second locations respectively on the elongate support, the first and second locations spaced apart from each other. Furthermore, at least one of the first and second arms may be attached to the elongate support for movement along the elongate support. The separation between the first and second arms may thus be changed to thereby change an extent of retraction achieved by way of engagement of the first and second anatomy engaging members with tissue.

The support and the arm may define cooperating surface profiles which provide for movement of the arm along the support. More specifically, the support may define a screw thread and the arm may define a threaded portion which engages with the screw thread. The support may therefore comprise a screw thread along at least part of its length and the arm may therefore comprise a threaded portion which engages with the screw thread of the support. The support may be of generally cylindrical form. Rotation of the support relative to the arm may drive the arm along the support by way of engagement of threaded portion and screw thread.

Where the first and second arms are movable along the support, the support may define a first screw thread and a second screw thread which extend along different parts of the support. The first screw thread may be in a first half of the support and the second screw thread may be in a second half of the support. The first and second screw threads may run in opposite directions such that each screw thread runs towards its respective end of the support or each screw thread runs way from its respective end of the support.

The first arm may define a first threaded portion and the second arm may define a second threaded portion, the first threaded portion engaging with the first screw thread and the second threaded portion engaging with the second screw thread. Rotation of the support relative to the first and second arms may cause the first and second screw threads to run in opposite directions. When the support is rotated in a first direction the first and second arms may be driven further apart to increase an extent of retraction and when the support is rotated in a second, opposite direction the first and second arms may be driven closer together to reduce an extent of retraction.

At least one of the first and second arms may comprise a proximal portion comprising the respective threaded portion. The proximal portion may be configured to encircle the support. The arm may further comprise an elongate arm member which extends from the proximal portion. The respective anatomy engaging member may depend from the arm member. The arm member may be fixedly attached to the proximal portion. More specifically, the arm member and the proximal portion may be integrally formed.

As described above, change in separation between the first and second arms may be achieved by rotation of the support. In use, the support may be rotated by the clinician. The clinician may use a tool to rotate the support. The support may therefore be shaped to interengage with a tool. More specifically, the support may define, such as at an end of the support, one of a plug and a socket which interengages with the other of a plug and a socket defined by the tool. The tool may comprise a handle which is gripped by the clinician when rotating the support by way of the tool. The handle may be comprised in an end of the tool opposite an end of the tool at which the plug and a socket is defined. The surgical retractor may comprise the tool.

At least one part of the surgical retractor may be formed from a plastics material. The plastics material may comprise at least one of the materials described above in respect of the anatomy engaging member. The surgical retractor may therefore be suited to single use.

According to a second aspect of the present invention there is provided a method of surgery on a human or animal subject involving use of a surgical retractor which comprises a support, first and second arms extending from a same side of the support, and first and second anatomy engaging members depending along their length from the first and second arms respectively, the first anatomy engaging member having a first interior face and a first exterior face, the second anatomy engaging member having a second interior face and a second exterior face, the first and second anatomy engaging members depending from the first and second arms respectively whereby the first exterior face faces away from the second anatomy engaging member and the second exterior face faces away from the first anatomy engaging member, and the first and second arms are spaced apart from each other along the support, the method of surgery comprising:
disposing the surgical retractor relative to an incision or wound in the human or animal subject and such that the first and second anatomy engaging members are received in the incision or wound, wherein
each of the first and second anatomy engaging members is formed from a plastics material,
each of the first exterior face and the second exterior face has a coating thereon, the coating comprising a haemostatic agent,
the first exterior face bears against first tissue at one side of the incision or wound and the second exterior face bears against second tissue at another side of the incision or wound, and
at least one of the first and second tissue is drawn back by at least one of the first and second anatomy engaging members.

The method of surgery on the human or animal subject is practiced by a clinician, such as a surgeon. The clinician disposes the surgical retractor relative to an incision or wound in the human or animal subject and such that the first and second anatomy engaging members are received in the incision or wound.

The method comprises at least one of the first and second tissue being drawn back, i.e. retracted, by the increase in separation. Whether one, other or both of the first and second tissue is retracted may depend on the nature of the anatomy involved. For example, both of the first and second tissue may be retracted where the first and second tissue is relatively yielding. By way of another example, one only of the first and second tissue may be retracted where one of the first and second tissue is more yielding, such a skin tissue, and the other of the first and second tissue is unyielding, such as muscle tissue.

The surgical retractor may be configured for change in separation between the first and second arms whereby separation between the first and second exterior faces is changed. The method may therefore further comprise operating the surgical retractor to increase separation between the first and second arms to thereby increase separation between the first and second exterior faces to an extent sufficient for the first exterior face to bear against first tissue at one side of the incision or wound and for the second exterior face to bear against second tissue at another side of the incision or wound. In view of there being haemostatic agent on the first and second exterior faces, the haemostatic agent is between the exterior faces and the tissue being borne against. The step of operating the surgical retractor to increase separation between the first and second arms may be carried out after the surgical retractor has been disposed appropriately relative to the incision or wound.

When the need for retraction has passed, the method may further comprise removal of the surgical retractor from the incision or wound, such as by the clinician.

Where the surgical retractor is configured for change in separation between the first and second arms, the method may comprise operating the surgical retractor, such as by a clinician, to decrease separation between the first and second arms to thereby decrease separation between the first and second exterior faces of the anatomy engaging members. The surgical retractor may then be removed from the incision or wound.

Further embodiments of the second aspect of the present invention may comprise one or more features of the first aspect of the present invention.

According to a third aspect of the present invention, there is provided a method of making a surgical retractor comprising a support, first and second arms, and first and second anatomy engaging members, each of the first and second anatomy engaging members formed from a plastics material, the first anatomy engaging member having a first interior face and a first exterior face, the second anatomy engaging member having a second interior face and a second exterior face, the method comprising:
assembling the surgical retractor such that the first and second arms extend from a same side of the support and are spaced apart from each other along the support;
assembling the surgical retractor such that the first anatomy engaging member depends along its length from the first arm whereby the first exterior face faces away from the second anatomy engaging member, and such that the second anatomy engaging member depends along its length from the second arm whereby the second exterior face faces away from the first anatomy engaging member; and
applying a coating to each of the first exterior face and the second exterior face, the coating comprising a haemostatic agent.

The coating may be applied to each of the first exterior face and the second exterior face before the step of assembling the surgical retractor such that the first anatomy engaging member depends along its length from the first arm, and such that the second anatomy engaging member depends along its length from the second arm. Application of the coating may be in accordance with at least one of the approaches described above with reference to the first aspect of the invention.

Further embodiments of the third aspect of the present invention may comprise one or more features of the first or second aspect of the present invention.

### BRIEF DESCRIPTION OF DRAWINGS

Further features and advantages of the present invention will become apparent from the following specific description, which is given by way of example only and with reference to the accompanying drawings, in which:

FIG. 1 is a perspective view of a surgical retractor according to an embodiment of the present invention; and

FIG. 2 is a flow chart representation of a method of surgery involving use of the surgical retractor of FIG. 1.

### DESCRIPTION OF EMBODIMENTS

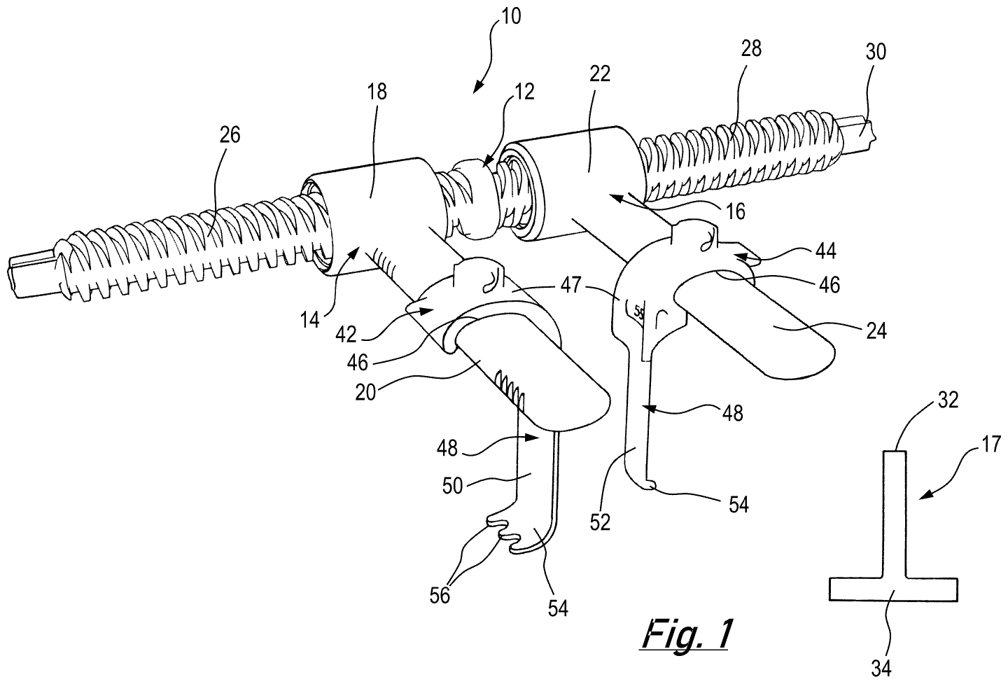

A perspective view of a surgical retractor 10 according to an embodiment of the present invention is shown in FIG. 1. The surgical retractor 10 comprises an elongate support member 12, a first arm 14, and a second arm 16. Each of the first arm 14 and the second arm 16 is mounted for movement along the support member 12 as described further below. The surgical retractor 10 further comprises a tool 17 (in FIG. 1 the tool 17 is not to scale with the rest of the surgical retractor 10).

The first arm 14 comprises a proximal portion 18 which encircles the support member 12 and an elongate first arm member 20 which extends from the proximal portion 18. The first arm member 20 is integrally formed with the proximal portion 18. The second arm 16 comprises a proximal portion 22 which encircles the support member 12 and an elongate second arm member 24 which extends from the proximal portion 22. The second arm member 24 is integrally formed with the proximal portion 22. As can be seen from FIG. 1, the first and second arm members 20, 24 are aligned with each other. Each of the first and second arm members is substantially elliptical in cross-section. The width in cross section of each arm member is 15 mm and the height in cross section is 10 mm.

One half of the exterior of the support member 12 defines a first screw thread 26 and the other half of the exterior of the support member 12 defines a second screw thread 28. The first and second screw threads 26, 28 run in opposite directions to each other such that each runs towards its respective end of the support member 12. Each of the first and second screw threads 26, 28 has four starts. The interior of each of the two proximal portions 18, 22 defines a threaded portion which interengages with a respective one of the first and second screw threads 26, 28.

Rotation of the support member 12 about its longitudinal axis moves each of the first and second arms 14, 16 in opposite directions along the support member 12 (i.e. together or apart depending on direction of rotation of the support member) by virtue of cooperation of the threaded portion on each proximal portion 18, 22 with a respective one of the first and second screw threads 26, 28.

As described above, the surgical retractor 10 further comprises a tool 17. An end of the support member 12 defines a plug 30 which extends in the longitudinal direction of the support member. The tool 17 is removably attached to the support member 12 by reception of the plug 30 of the support member in a socket 32 defined in a first end of the tool. The plug 30 and socket 32 are shaped to interlock with each other. The tool 17 comprises a hand grippable portion 34 at the second, opposite end thereof. When the plug 30 is received in the socket 32, the clinician rotates the tool 17 by way of the hand grippable portion 34. Rotation of the tool 17 is transmitted to the support member 12 by way of the interlocked plug 30 and socket 32. As described above, rotation of the support member 12 moves each of the first and second arms 14, 16 in opposite directions along the support member 12 whereby separation between the first and second arms changes.

The surgical retractor 10 further comprises first and second anatomy engaging members 42, 44. The first and second anatomy engaging members 42, 44 are of the same form and function. As can be seen from FIG. 1, the first anatomy engaging member 42 is attached to the first arm 18 and the second anatomy engaging member 44 is attached to the second arm 22 whereby each anatomy engaging member depends substantially orthogonally from its respective arm.

Each of the first and second anatomy engaging members 42, 44 defines an engaging member profile 46 at its proximal end whereby the anatomy engaging member is clipped to and thereby releasably engaged with an arm member 20, 24 while allowing for sliding movement of the anatomy engaging member along the arm member when so attached. The engaging member profile 46 is defined by an attachment portion 47 of the anatomy engaging member. The shape of the engaging member profile 46 corresponds to the profile of the arm member 20, 24 whereby the engaging member profile abuts against the arm member. The engaging member profile 46 has a main profile portion which extends across the upper surface of the arm member 20, 24. The engaging member profile 46 also has first and second lateral portions each at a respective end of the main profile portion. The first and second lateral portions are each shaped to extend over a side of the arm member 20, 24 and then a short distance across the lower surface of the arm member. The distal end of the first lateral portion therefore opposes the distal end of the second lateral portion with the distal ends of the first and second lateral portions being spaced apart from each other across the lower surface of the arm member 20, 24.

The anatomy engaging member 42, 44 is attached to the arm member 20, 24 by sliding the engaging member profile 46 over the distal end of the arm member. Alternatively and in view of the anatomy engaging member 42, 44 being formed from a resilient material, the attachment portion 47 of the anatomy engaging member is pressed against the upper surface of the arm member whereby the distal ends of the first and second lateral portions are forced apart against the bias exerted by the resilient material to an extent sufficient to admit the arm member to the space defined by the engaging member profile 46. When the arm member is admitted properly in the space defined by the engaging member profile 46, the distal ends of the first and second lateral portions move together again under bias exerted by the resilient material whereby the anatomy engaging member 42, 44 is attached securely to the arm member 20, 24 whilst allowing for movement of the anatomy engaging member along the arm member.

Each of the first and second anatomy engaging members 42, 44 has a main body 48 which is joined at its proximal end to the attachment portion 47. The main body 48 has the form of a substantially rectangular plate which defines a curve across its width. An exterior face 50 of the main body 48 is convex and an interior face 52 of the main body is concave. Each of the first and second anatomy engaging members 42, 44 defines a foot portion 54 at a distal end thereof. The foot portion 54 extends generally orthogonally to the distal end of the main body 48. Each foot portion 48 defines two recesses 56 at a distal end thereof with the two recesses being spaced apart in the transverse direction, i.e. across the width of the anatomy engaging member.

The retractor 10 is formed substantially completely from GS Ixef® 1022 from Solvay S. A. Parts of the retractor are integrally formed by injection moulding. For example, the first and second arms 14, 16 are each integrally formed. By way of another example, the support member 12 is integrally formed. By way of further example, the tool 17 is integrally formed. The tool 17 is formed from polycarbonate (PC). More specifically, the tool 17 is formed from Makrolon® 2458 from Bayer MaterialScience AG, 51368 Leverkusen, Germany.

The main body 48 of each of the first and second anatomy engaging members 42, 44 has a coating of at least one haemostatic agent. The at least one haemostatic agent is at least one of: a clay mineral, such as an aluminosilicate mineral; a polysaccharide, such as chitosan; an astringent, such as anhydrous aluminium sulfate; and a coagulation factor, such as thrombin.

Before the coating is applied, as described further below, the main body 48 of each of the first and second anatomy engaging members 42, 44 is subject to oxygen, argon, or air plasma treatment to increase surface roughness and thereby promote adhesion of the subsequently applied coating.

According to one coating application approach and depending on the nature of the haemostatic agent and how the haemostatic coating is to be applied to the anatomy engaging member, the haemostatic agent, such as chitosan, is dissolved in an appropriate solvent or is dispersed in an appropriate medium of suspension to form a colloid to form a haemostatic mixture. The haemostatic mixture is applied to the anatomy engaging member to form the haemostatic coating by spraying the anatomy engaging member with the haemostatic mixture or by immersion of the anatomy engaging member in the haemostatic mixture. The thus applied coating on the anatomy engaging member is then dried, for example by heating the anatomy engaging member at between 60 to 80 degrees Centigrade.

According to another coating application approach, and depending on the nature of the haemostatic agent, the haemostatic coating is deposited on the anatomy engaging member by hot or cold plasma spraying or by chemical vapor deposition.

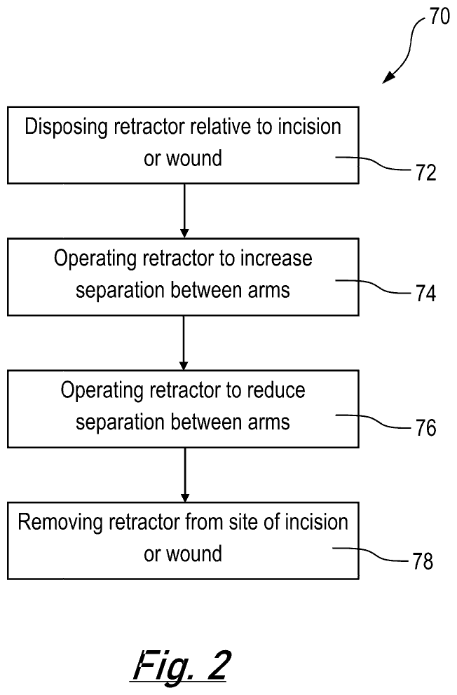

A flow chart representation of a method of surgery 70 involving use of the surgical retractor 10 of FIG. 1 is shown in FIG. 2. The method of surgery 70 comprises disposing the surgical retractor 10 relative to an incision or wound in a human or animal subject and such that the first and second anatomy engaging members 42, 44 are received in the incision or wound 72. The surgical retractor 10 is operated by rotation of the support member 12 such that the first and second anatomy engaging members 42, 44 are sufficiently close to each other that the first and second anatomy engaging members can be received readily in the incision or wound.

The clinician then operates the surgical retractor 10 by means of the tool 17 to rotate the support member 12 to thereby increase separation between the first and second arms 14, 16, 74. This increases separation between the first and second exterior faces 50 to an extent sufficient for the first exterior face to bear against and retract first tissue at one side of the incision or wound and for the second exterior face to bear against and retract second tissue at another side of the incision or wound.

Depending on the application, the foot portions 54 fit underneath anatomy whereby the anatomy is gathered up and held by the foot portion. The foot portion 54 is useful for engaging with a substantial mass of anatomy, such as muscle tissue, or perhaps even for fitting underneath or resting upon bone. When the surgical retractor 10 is in-situ, the haemostatic agent or agents are operative to promote haemostasis.

When need for the surgical retractor 10 has passed, the clinician operates the surgical retractor 10 to reduce separation between the arms 14, 16 to sufficient extent to allow for ease of removal of the first and second anatomy engaging members 42, 44 from the incision or wound 76. The surgical retractor 10 is then removed by the clinician from the site of the incision or wound 78.

1. A surgical retractor comprising:
a support;
a first arm extending from the support;
a second arm extending from the support, the first and second arms extending from a same side of the support and spaced apart from each other along the support;
a first anatomy engaging member having a first interior face and a first exterior face; and
a second anatomy engaging member having a second interior face and a second exterior face, wherein
the first anatomy engaging member depends along its length from the first arm whereby the first exterior face faces away from the second anatomy engaging member;
the second anatomy engaging member depends along its length from the second arm whereby the second exterior face faces away from the first anatomy engaging member;
each of the first and second anatomy engaging members is formed from a plastics material; and
each of the first exterior face and the second exterior face has a coating thereon, the coating comprising at least one haemostatic agent.

2. The surgical retractor according to claim 1, in which the first and second interior faces have thereon a coating comprising at least one haemostatic agent.

3. The surgical retractor according to claim 1, in which the coating comprises a polysaccharide as the haemostatic agent.

4. The surgical retractor according to claim 3, in which the polysaccharide comprises chitosan.

5. The surgical retractor according to claim 1, in which the coating comprises plural different haemostatic agents, the plural different haemostatic agents stimulating haemostasis in respectively different ways.

6. The surgical retractor according to claim 1, in which the coating comprises at least one of a starch and a clay mineral as the haemostatic agent.

7. The surgical retractor according to claim 6, in which the clay mineral belongs to one of the kaolin group and the smectite group.

8. The surgical retractor according to claim 1, in which the coating comprises at least one astringent as the haemostatic agent.

9. The surgical retractor according to claim 1, in which each of the first and second anatomy engaging members comprises a main body which defines the respective exterior and interior faces, the main body having the form of an elongate plate.

10. The surgical retractor according to claim 1, in which at least one of the first and second anatomy engaging members is attached to the respective arm for movement of the anatomy engaging member along the arm.

11. The surgical retractor according to claim 1, in which each of the first and second anatomy engaging members is configured such that it extends substantially orthogonally to the respective arm.

12. (canceled)

13. The surgical retractor according to claim 1, in which each of the first and second anatomy engaging members is formed from polyarylamide.

14. The surgical retractor according to claim 1, in which the support is elongate and the first and second arms extend from respective locations on the elongate support, the respective locations spaced apart from each other along the longitudinal direction of the elongate support.

15. The surgical retractor according to claim 14, in which at least one of the first and second arms is attached to the elongate support for movement along the elongate support.

16. The surgical retractor according to claim 15, in which the support is of generally cylindrical form and defines a screw thread along at least part of its length, and the arm defines a threaded portion which engages with the screw thread, whereby rotation of the support relative to the arm drives the arm along the support by way of engagement of the threaded portion and the screw thread.

17. The surgical retractor according to claim 1, in which the support, and the first and second arms are formed from a plastics material.

18. A method of making a surgical retractor comprising a support, first and second arms, and first and second anatomy engaging members, each of the first and second anatomy engaging members formed from a plastics material, the first anatomy engaging member having a first interior face and a first exterior face, the second anatomy engaging member having a second interior face and a second exterior face, the method comprising:
assembling the surgical retractor such that the first and second arms extend from a same side of the support and are spaced apart from each other along the support;
assembling the surgical retractor such that the first anatomy engaging member depends along its length from the first arm whereby the first exterior face faces away from the second anatomy engaging member, and such that the second anatomy engaging member depends along its length from the second arm whereby the second exterior face faces away from the first anatomy engaging member; and
applying a coating to each of the first exterior face and the second exterior face, the coating comprising at least one haemostatic agent.

19. The method according to claim 18 further comprising:
forming a haemostatic mixture by one of: a) dissolving the at least one haemostatic agent in a solvent, and b) dispersing the at least one haemostatic agent in a medium of suspension to form a colloid; and in which
the step of applying the coating to each of the first and second exterior faces comprises spraying the haemostatic mixture on the first and second exterior faces.

20. The method according to claim 19, in which the haemostatic mixture is sprayed on the first and second exterior faces by one of hot plasma spraying and cold plasma spraying.

21. The method according to claim 18 further comprising plasma treatment of the first and second exterior faces to increase their surface roughness, the step of plasma treatment carried out before the step of applying the coating to each of the first and second exterior faces.
